(19) United States
(12) Patent Application Publication
(10) Pub. No.: US 2024/0033528 A1
(43) Pub. Date: Feb. 1, 2024

(54) IMPLANTABLE PULSE GENERATOR FOR PROVIDING A NEUROSTIMULATION THERAPY BY BLENDING CURRENT AND VOLTAGE CONTROL FOR OUTPUT AND METHODS OF OPERATION

(71) Applicant: ADVANCED NEUROMODULATION SYSTEMS, INC., PLANO, TX (US)

(72) Inventors: Daran DeShazo, Lewisville, TX (US); Christopher Palmer, Plano, TX (US)

(21) Appl. No.: 18/350,010

(22) Filed: Jul. 11, 2023

Related U.S. Application Data
(60) Provisional application No. 63/393,342, filed on Jul. 29, 2022.

(51) Int. Cl. A61N 1/36

(52) U.S. Cl. CPC A61N 1/36167; A61N 1/36157; A61N 1/36153

(57) ABSTRACT

System and methods for providing a pulsed therapy comprises an implantable pulse generator (IPG) having circuitry configured to control generation and delivery of electrical pulses using at least one electrode of a stimulation lead. The IPG further comprises memory configured to store program instructions and one or more processors configured to execute the program instructions. The one or more processors are configured to deliver the pulses, having a corresponding pulse duration and an amplitude, to the at least one electrode. The amplitude can vary over the pulse duration of at least one of the pulses. The processor(s), monitor a signal indicative of a present level of the amplitude of the at least one pulse, and based on the signal, declare a pulse valid based on the present level satisfying an initial criteria for at a sub-duration of the pulse duration that is less than an entire pulse duration.

### CROSS-REFERENCE TO RELATED APPLICATIONS

This application claims priority to U.S. Provisional Patent Application No. 63/393,342, filed 29 Jul. 2022, entitled “IMPLANTABLE PULSE GENERATOR FOR PROVIDING A NEUROSTIMULATION THERAPY BY BLENDING CURRENT AND VOLTAGE CONTROL FOR OUTPUT AND METHODS OF OPERATION”, the subject matter of the which is incorporated herein by reference in its entirety.

### BACKGROUND

Implantable medical devices have improved how medical care is provided to patients with certain types of chronic illnesses and disorders. For example, implantable neurostimulator devices can provide pain reduction for chronic pain patients and reduce motor difficulties in patients with Parkinson's disease and other movement disorders. A variety of other medical devices are proposed and are in development to treat other disorders in a wide range of patients.

Neural activity in the brain can be influenced by electrical energy that is supplied from a stimulation pulse generator or other waveform generator, Various patient perceptions and/or neural functions can be promoted or disrupted by applying an electrical or magnetic signal to the brain. Medical researchers and clinicians have attempted to manage various neurological conditions using electrical or magnetic stimulation signals to control or affect brain functions. For example, Deep Brain Stimulation (DBS) may reduce some of the symptoms associated with Parkinson's Disease, which results in movement or muscle control problems and is debilitating to a great number of individuals worldwide.

A stimulation system pulse generator may be provided in various configurations, such as an implanted pulse generator (IPG). A typical IPG system configuration comprises of a surgically implanted, internally-powered pulse generator and multi-electrode lead. The implanted pulse generator may commonly be encased in a hermetically sealed housing and surgically implanted, for example, in a subclavicular, upper chest, or lower back location. An electrode assembly may be implanted to deliver stimulation signals to a stimulation site. The electrode assembly is coupled to the pulse generator via biocompatible and insulated lead wires. A power source, such as a battery, is contained within the housing of the pulse generator.

Brain anatomy typically requires precise targeting of tissue for stimulation by deep brain stimulation systems. For example, deep brain stimulation for Parkinson's disease commonly targets tissue within or close to the subthalamic nucleus (STN). The STN is a relatively small structure with diverse functions. Stimulation of undesired portions of the STN or immediately surrounding tissue can result in undesired side effects. For example, muscle contraction or muscle tightening may be caused by stimulation of neural tissue that is near the STN. Mood and behavior dysregulation and other psychiatric effects have been reported from undesired stimulation of neural tissue near the STN in Parkinson's patients.

To avoid undesired side effects in deep brain stimulation, neurologists often attempt to identify a particular electrode for stimulation that only stimulates the neural tissue associated with the symptoms of the underlying disorder while minimizing use of electrodes that stimulate other tissue. Also, neurologists may attempt to control the pulse amplitude, pulse width, and pulse frequency to limit the stimulation field to the desired tissue.

As an improvement over conventional deep brain stimulation leads, leads with segmented electrodes have been proposed. Conventional deep brain stimulation leads include electrodes that fully circumscribe the lead body. Leads with segmented electrodes include electrodes on the lead body that only span a limited angular range of the lead body. As used herein, the term “segmented electrode” refers to an electrode or a group of electrodes that are positioned at approximately the same longitudinal location along the longitudinal axis of a lead and that are angularly positioned about the longitudinal axis so they do not overlap and are electrically isolated from one another. For example, at a given position longitudinally along the lead body, three electrodes can be provided with each electrode covering respective segments of less than 120 degrees about the outer diameter of the lead body. By selecting between such electrodes, the electrical field generated by stimulation pulses or waveforms can be more precisely controlled and, hence, stimulation of undesired tissue—which often causes detrimental therapy side effects—can be more easily avoided. This has particular benefit for improved stimulation therapy efficacy if the DBS lead is slightly misplaced from the target area in the brain during surgical implantation.

Implanted medical devices may estimate the impedance of the patient load by taking a voltage or current measurement during a sub-perception stimulation pulse or at the end of a therapy stimulation pulse. If the implanted medical device delivers a controlled voltage, then a current measurement can be used to estimate impedance. If the device delivers a controlled current, then a voltage measurement can be used to estimate impedance. In either of these cases, a single measurement provides only an estimation of a resistance since there is no time-dependent information in the measurement.

Neuromodulation systems have conventionally used either current-controlled or voltage-controlled technology for the output pulse. In some cases, advantages may be realized by delivering current-controlled pulses as a better prediction of the charge being delivered can be made. A programmable voltage multiplier is often used to provide the supply voltage for current-controlled pulses. Using the programmable multiplier, battery current can be saved by using the lowest voltage setting possible; however, the design also limits the maximum available output voltage.

Electrode geometries have a direct impact on the impedance a stimulation system pulse generator must overcome to deliver a current-controlled pulse. Smaller electrodes increase impedance overall due to the increase in rate of charging the small capacitance of a small-geometry electrode such as may be included on a segmented lead.

Accordingly, a need remains for methods and devices that can improve battery usage efficiency while providing the desired total energy to meet the demands of high energy therapy using small-geometry electrodes.

### BRIEF DESCRIPTION OF THE DRAWINGS

FIG. 1 depicts a neurostimulation system that is adapted according to an example embodiment and is shown as a high-level functional block diagram in accordance with embodiments herein.

FIG. 2 depicts a system in which an implanted medical device may be programmed and/or monitored by a programmer device to provide therapy in accordance with embodiment herein.

FIG. 3 a cathode-side programmable amplitude current regulator that can be used in some embodiments.

FIG. 4 depicts a model of the electrical interaction between a patient's tissue and stimulation electrodes.

FIG. 5 depicts a current regulator circuit configured to output a current-controlled output and an amplitude monitoring signal in accordance with embodiments herein.

FIG. 6A illustrates a method for delivering and monitoring pulses to ensure that a sub-duration of the entire pulse duration maintains a desired amplitude in accordance with embodiments herein.

FIG. 6B illustrates a set of graphs showing different pulses at different amplitudes in accordance with embodiments herein.

FIG. 7A illustrates a method for delivering and monitoring pulses while allowing the amplitude of the pulse to decrease or clip while maintaining the input voltage from the voltage multiplier in accordance with embodiments herein.

FIG. 7B illustrates another set of graphs showing different pulses having different amplitudes in accordance with embodiments herein.

FIGS. 8A and 8B respectively depict stimulation portions for inclusion at the distal end of a stimulation lead according to example embodiments.

FIG. 9 depicts a paddle lead stimulation portion for inclusion at the distal end of a stimulation lead according to example embodiments.

FIG. 10 is a plot illustrating components of a voltage load across the model shown in FIG. 4 as a result of a constant-current therapy pulse.

FIG. 11 depicts a circuit that can be used for the complex impedance measurements of an electrode/tissue interface.

FIG. 12 shows example pulse widths that can be achieved at different current amplitudes (mA) for an impedance of an electrode in accordance with embodiments herein.

### SUMMARY

In accordance with embodiments herein, an implantable pulse generator (IPG) has circuitry configured to control generation and delivery of electrical pulses using at least one electrode of a stimulation lead. The IPG further comprises memory configured to store program instructions and one or more processors configured to execute the program instructions. The one or more processors are configured to deliver the pulses to the at least one electrode. The pulses have a corresponding pulse duration and an amplitude. The amplitude varies over the pulse duration of at least one of the pulses. The one or more processors monitor a signal indicative of a present level of the amplitude of the at least one pulse, and based on the signal, declare a valid pulse based on the present level satisfying an initial criteria for a sub-duration of the pulse duration that is less than an entire pulse duration.

Optionally, the IPG further comprises a current regulator circuit configured to output the signal, wherein the signal is indicative of the present level of current. Optionally, the signal is indicative of the present level of voltage.

Optionally, the circuitry further comprises a voltage multiplier configured to generate the pulses. The one or more processors are further configured to direct the voltage multiplier to increase a voltage level utilized for generating the electrical pulses responsive to the present level not satisfying the initial criteria.

Optionally, the initial criteria includes at least one threshold level associated with the amplitude of the pulse, and the program instructions further comprise instructions for causing the one or more processors to, responsive to the present level of the amplitude of the pulse being less than the at least one threshold level for at least a portion of the sub-duration of the pulse duration, declare the pulse invalid, and, responsive to the present level of the amplitude of the pulse being equal to or greater than the at least one threshold level for at least a portion of the sub-duration of the pulse duration, declare the pulse valid.

Optionally, wherein the pulses include a first pulse that satisfies the initial criteria for the entire pulse duration, a second pulse that satisfies the initial criteria for the sub-duration but not for the entire pulse duration, and a third pulse that does not satisfy the initial criteria for the sub-duration. The one or more processors are configured to declare the first and second pulses to be valid and the third pulse to be invalid. Optionally, the circuitry further comprises a power supply coupled to at least one of a voltage multiplier or a control circuit. The power supply and the at least one of the voltage multiplier or control circuit are configured to generate the first, second and third pulses.

Optionally, the instructions to monitor the signal further comprise instructions to monitor the signal for the sub-duration of the pulse duration. The sub-duration is determined based on a predetermined number of clock signals or a predetermined time duration. Optionally, the IPG is a neurostimulation device and the pulses represent a neurostimulation therapy. Optionally, the program instructions further comprise instructions for causing the one or more processors to, responsive to the pulse being declared valid, not set an error message, and not adjust a voltage level utilized for generating the electrical pulses.

Optionally, the program instructions further comprise instructions for causing the one or more processors to, based on the signal, determine whether a voltage level utilized for generating the electrical pulses is equal to or above a voltage threshold value. The one or more processors, responsive to the voltage level being equal to or above the voltage threshold value, determine whether an auto-reduce feature is disabled, wherein the auto-reduce feature, when disabled, is configured to provide an indication that the voltage level is not to be reduced. The one or more processors, responsive to the auto-reduce feature being disabled, do not adjust the voltage level utilized for generating the electrical pulses.

In accordance with embodiments herein, a computer implemented method for providing a pulsed therapy comprises, under control of one or more processors, in an implantable pulse generator (IPG), configured with executable instructions, delivering electrical pulses to at least one electrode interconnected with the IPG. The pulses have a corresponding pulse duration and an amplitude, and the amplitude varies over the pulse duration of at least one of the pulses. The method monitors a signal indicative of a present level of the amplitude of the at least one pulse. Based on the signal, the method declares a valid pulse based on the present level satisfying an initial criteria for a sub-duration of the pulse duration that is less than an entire pulse duration.

Optionally, the signal is indicative of the present level of current output by a current regulator circuit of the IPG. Optionally, the signal is indicative of the present level of voltage.

Optionally, the method further comprises declaring an invalid pulse based on the present level not satisfying the initial criteria for a portion of the sub-duration of the pulse duration. Responsive to the pulse being declared invalid, the method increases a voltage level utilized by a voltage multiplier for generating the pulses.

Optionally, the method further comprises declaring the pulse invalid in response to the present level of the amplitude of the pulse being less than at least one threshold level for a portion of the sub-duration of the pulse duration, wherein the at least one threshold level is associated with the amplitude of the pulse. The method further comprises responsive to the present level of the amplitude of the pulse being equal to or greater than the at least one threshold level for the sub-duration of the pulse duration, declaring the pulse valid.

Optionally, the pulses include a first pulse that satisfies the initial criteria for the entire pulse duration, a second pulse that satisfies the initial criteria for the sub-duration but not for the entire pulse duration, and a third pulse that does not satisfy the initial criteria for the sub-duration, and the method declares the first and second pulses to be valid and the third pulse to be invalid. Optionally, the method further comprises generating, utilizing a power supply coupled to at least one of a voltage multiplier or a control circuit, the first, second and third pulses.

Optionally, the method further comprises, responsive to the pulse being declared valid, not setting an error message and maintaining a voltage level utilized for generating the electrical pulses.

Optionally, the method further comprises, based on the signal, determining whether a voltage level utilized for generating the electrical pulses is equal to or above a voltage threshold value. Responsive to the voltage level being equal to or above the voltage threshold value, the method determines whether an auto-reduce feature is disabled, wherein the auto-reduce feature, when disabled, is configured to provide an indication that the voltage level in not to be reduced. Responsive to the auto-reduce feature being disabled, the method maintains the voltage level utilized for generating the electrical pulses.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

### Terms

The term “implantable pulse generator”, “IPG”, and “implantable neurostimulator” shall mean an implantable medical device that delivers pulses, using one or more electrode of a lead connected to the IPG, to one or more tissue area within a patient.

The terms “clinician programmer”, “clinician programmer device” and more generally “device” shall mean a computer, application, smartphone, and the like utilized by a clinician to define or control a therapy provided to a patient by the IPD. The clinician(s) may define or set one or more therapy parameters, such as pulse amplitudes, pulse frequencies, pulse patterns, and/or a variety of other therapy parameters.

The terms “maximum output voltage”, “VMO”, and “compliance voltage” shall mean that a maximum available voltage level is output from a voltage supply or voltage multiplier. The compliance voltage can be a multiple of a battery voltage level associated with a battery within the IPG.

The terms “clip” and “clipping” shall mean that an amplitude level of a pulse delivered by the IPG decreases below a threshold level prior to an end of the entire pulse duration. The clipping can occur proximate a trailing edge of the pulse, and/or at any time during the pulse duration after the programmed amplitude has been achieved, extending a sub-duration of the pulse duration, at the leading edge of the pulse.

The term “auto-reduce feature” shall mean an option that in some cases can be implemented by the IPG to reduce the voltage level output from the voltage supply or voltage multiplier. In some embodiments, the auto-reduce feature may reduce the output voltage level when the pulse is clipped. In other embodiments, the auto-reduce feature is disabled, thus preventing the reduction of the output voltage level and/or maintaining the output voltage level.

The “amplitude monitoring signal” and more generally “signal” shall mean a signal that outputs a status or result based on a comparison of an amplitude level delivered to an electrode (or electrodes) to one or more threshold. A first output can be used to indicate that the amplitude level meets or exceeds one or more amplitude threshold levels for a sub-duration or for at least a portion of a sub-duration of a pulse duration (e.g., indicates a valid pulse) and a second output can be used to indicate that the amplitude level does not meet one or more amplitude threshold levels for a sub-duration or for at least a portion of a sub-duration of a pulse duration (e.g., indicates an invalid pulse).

Embodiments may be implemented in connection with one or more implantable medical devices (IMDs). In accordance with embodiments herein, the methods, devices, and systems may be implemented in connection with the methods, devices, and systems described in U.S. published application 2020/0346005, entitled “Neurostimulation method and system with current regulator biased by floating power supply”, published Nov. 5, 2020, and U.S. published application 2020/0324126, entitled “Wireless power transfer circuit for a rechargeable implantable pulse generator”, published Oct. 15, 2020, which are incorporated herein by reference in their entireties.

### System Overview

Embodiments disclosed herein describe systems and methods for monitoring that a full output current is delivered for a portion of the pulse duration (e.g., current-controlled behavior) while allowing the trailing edge of the pulse to drift into a voltage-controlled behavior. Allowing some portion of the pulse to be delivered without enforcing that the full amplitude (e.g., approximately 100 percent of the desired/programmed amplitude) is met for the entire pulse duration can i) extend the available output range, ii) improve battery usage efficiency, and/or iii) increase total energy delivered to meet the demands of high energy therapy.

Embodiments disclosed herein describe systems and methods for monitoring a signal that can indicate whether the present level of the amplitude of the pulse satisfies an initial criteria for a sub-duration of the entire pulse duration. When the initial criteria is not satisfied for the sub-duration, an output of the voltage supply can be increased if the voltage supply is not at or near a maximum voltage setting.

In some cases, an auto-reduce feature can be disabled, allowing therapy to continue when the amplitude of the pulse decreases in a latter portion or trailing edge of the pulse.

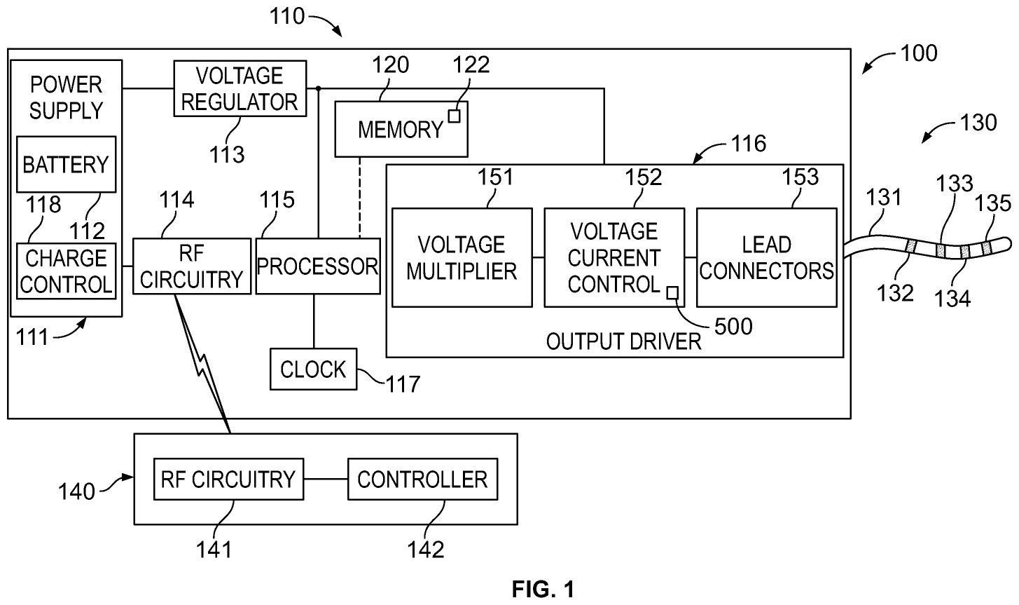

FIG. 1 depicts a neurostimulation system 100 that is adapted according to an example embodiment and is shown as a high-level functional block diagram. Neurostimulation systems are devices that generate electrical pulses and deliver the pulses to neural tissue of a patient to treat a variety of disorders. As noted above, a neurostimulation system 100 may be used to provide DBS therapy for patients with movement disorders. Neurostimulation system 100 may also provide Spinal Cord Stimulation (SCS) in which electrical pulses are delivered to neural tissue of the spinal cord for the purpose of chronic pain control. While a precise understanding of the interaction between the applied electrical energy and the neural tissue is not fully appreciated, it is known that application of an electrical field to spinal neural tissue can effectively inhibit certain types of pain transmitted from regions of the body associated with the stimulated neural tissue to the brain.

Neurostimulation systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. For SCS therapy, the distal end of a respective stimulation lead is implanted within the epidural space to deliver the electrical pulses to the appropriate nerve tissue within the spinal cord. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

Neurostimulation system 100 of the illustrated embodiment includes a generator portion, shown as implantable pulse generator (IPG) 110, for providing a stimulation or energy source, a stimulation portion, shown as lead 130, for application of the stimulus pulse(s), and an optional external controller, shown as programmer/controller 140, to program and/or control IPG 110 via a wireless communications link. IPG 110 may be implanted within a living body (not shown) for providing electrical stimulation from IPG 110 to a selected area of the body, such as a region of the brain or spinal cord, via lead 130. For example, the IPG 110 can be a neurostimulation device and the pulses can represent a neurostimulation therapy. In some embodiments, IPG 110 provides electrical stimulation under control of external programmer/controller 140. It should be appreciated that, although lead 130 is illustrated to provide a stimulation portion of stimulation system 100 and is configured to provide stimulation remotely with respect to the generator portion 110 of stimulation system 100, a lead 130 as described herein is intended to encompass a variety of stimulation portion configurations. Furthermore, a lead configuration may include more (e.g., 8, 16, 32, etc.) or fewer (e.g., 1, 2, etc.) electrodes than those represented in the illustrations.

IPG 110 of the illustrated embodiment includes power supply 111, voltage regulator 113, RF circuitry 114, one or more processor (e.g., microcontroller, microprocessor, etc.) 115, output driver circuitry 116, and clock 117, as are described in further detail below. Power supply 111 provides a source of power, such as from battery 112, to other components of IPG 110, and may be regulated by voltage regulator 113. Battery 112 may comprise a non-rechargeable (e.g., single use) battery, a rechargeable battery, a capacitor, and/or like power sources. Charge control 118 provides management for battery 112 and power supply 111 in some embodiments. In some embodiments, the entire IPG 110 device may need to be accessed by a surgical procedure to replace battery 112. In other embodiments, when battery 112 is depleted, it may be recharged after being implanted, for example, inductive coupling and external charging circuits. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

RF circuitry 114 provides data communication between processor 115 and controller 142 in external programmer/controller 140, via RF circuitry 141. It should be appreciated that RF circuitry 114 and/or 141 may be a receiver, a transmitter, and/or transceiver depending upon the communication links desired using far-field and/or near field communication communications. The communication links may be established using suitable communication methods such as inductive wireless communication, low energy BLUETOOTH® communication, and medical band wireless communication as examples. An example of BLUETOOTH® communication between an implantable medical device and a programmer device is found, for example, in U.S. Pat. No. 9,894,691, entitled SYSTEMS AND METHODS FOR ESTABLISHING A COMMUNICATION LINK BETWEEN AN IMPLANTABLE MEDICAL DEVICE AND AN EXTERNAL INSTRUMENT, the disclosure of which is incorporated herein by reference.

The one or more processor 115 provides control with respect to the operation of IPG 110, such as in accordance with a program provided thereto by external programmer/controller 140. Software code is typically stored in one or more memory 120 of IPG 110 for execution by the processor 115 to control the various components of the device. The software code (e.g., program instructions) stored in memory 120 of IPG 110 may support operations of embodiments disclosed herein.

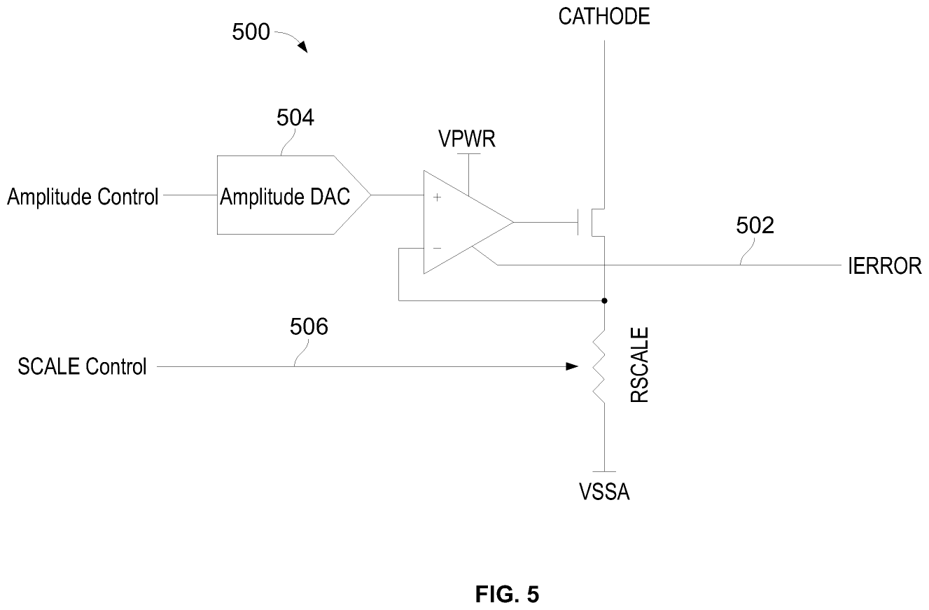

Output driver circuitry 116 generates and delivers pulses to selected ones of electrodes 132-135 on lead body 131 under control of processor 115. The pulses can have a corresponding pulse duration and an amplitude, and the amplitude can vary over the pulse duration. For example, voltage multiplier 151 and voltage/current control 152 (e.g., control circuit) may be controlled to deliver a blended current-controlled and voltage-controlled pulse using selected ones of electrodes 132-135. Current regulator circuit 500 can be interconnected with and/or integral with the voltage/current control 152 and is discussed further below in FIG. 5. The current regulator circuit 500 and/or the voltage/current control 152, one or both coupled to the power supply 111 and/or voltage multiplier 151, are configured to output a signal indicative of a present level of the amplitude of at least one pulse, and the signal can be indicative of the present level of current or voltage. Although the voltage multiplier 151 is discussed herein, it should be understood that other voltage supply and/or voltage supplies may provide the same functionality.

Clock 117 preferably provides system timing information, such as may be used by processor 115 in controlling system operation, as may be used by voltage multiplier 151 in generating a desired voltage, and as may be used to measure an amount of time when monitoring one or more signal that can indicate a particular state such as an error state (e.g., valid, invalid, etc.) and/or a relationship of an amplitude (e.g., amplitude of current or voltage) to one or more threshold level, etc. For example, the signal can be monitored for a sub-duration of an entire pulse duration, wherein the sub-duration is determined based on a predetermined number of clock signals or a predetermined time duration.

If the present level of the amplitude of the pulse is less than the at least one threshold level for at least a portion of the sub-duration of the pulse duration, the processor 115 can declare the pulse invalid, and if the present level of the amplitude of the pulse is equal to or greater than the at least one threshold level for at least a portion of the sub-duration of the pulse duration, the processor can declare the pulse valid. In some embodiments, the pulses can include a first pulse that satisfies the initial criteria for the entire pulse duration, a second pulse that satisfies the initial criteria for the sub-duration but not for the entire pulse duration, and a third pulse that does not satisfy the initial criteria for the sub-duration. In this case, the processor 115 can declare the first and second pulses to be valid and the third pulse to be invalid. Also, if the pulse is declared valid by the processor 115, an error message is not set and the voltage level utilized for generating the electrical pulses is not adjusted.

A status of an auto-reduce feature 122 can be stored in the memory 120. The processor 115 can determine whether a voltage level utilized for generating the electrical pulses is equal to or above a voltage threshold value. If the voltage level is equal to or above the voltage threshold value, the processor 115 can determine whether the status of the auto-reduce feature 122 is disabled, which provides an indication that the voltage level is not to be reduced. Therefore, if the auto-reduce feature 122 is disabled, the voltage level that is utilized for generating the electrical pulses is not adjusted.

Lead 130 of the illustrated embodiment includes lead body 131, preferably incorporating a plurality of internal conductors coupled to lead connectors (not shown) to interface with lead connectors 153 of IPG 110. Lead 130 further includes electrodes 132-135, which are preferably coupled to the internal conductors 153. The internal conductors 153 provide electrical connection from individual lead connectors to each of a corresponding one of electrodes 132-135. In the exemplary embodiment the lead 130 is generally configured to transmit one or more electrical signals from IPG 110 for application at, or proximate to, a spinal nerve or peripheral nerve, brain matter, muscle, or other tissue via electrodes 132-135. IPG 110 can control the electrical signals by varying signal parameters, such as pulse amplitude, pulse width, pulse frequency, burst frequency, and/or the like in order to deliver a desired therapy or otherwise provide operation as described herein.

Although the embodiment illustrated in FIG. 1 includes four electrodes, it should be appreciated that any number of electrodes, and corresponding conductors, may be utilized according to some embodiments. Moreover, various types, configurations and shapes of electrodes (and lead connectors) may be used according to some embodiments. An optional lumen (not shown) may extend through the lead 130, such as for use in delivery of chemicals or drugs or to accept a stylet during placement of the lead within the body. Additionally, or alternatively, the lead 130 (stimulation portion) and IPG 110 (generator portion) of stimulation system 100 may comprise a unitary construction, such as that of a microstimulator configuration.

In an embodiment, a programmable neurostimulation system 100 supplies suitable therapy pulses to a patient by enabling a pattern of electrical pulses to be varied across the electrodes 132-135 of a lead or leads 130. Such systems enable electrodes of a connected stimulation lead 130 to be set as an anode (+), as a cathode (−), or to a high-impedance state (OFF). As is well known, negatively charged ions flow away from a cathode toward an anode. Consequently, a range of very simple to very complex electrical fields can be created by defining different electrodes 132-135 in various combinations of (+), (−), and OFF. Of course, in any instance, a functional combination must include at least one anode and at least one cathode. In an embodiment, the case or “can” of the neurostimulation system 100 or IPG 110 may function as an anode. When determining the appropriate electrode configurations, the selection of electrodes 132-135 to function as anodes can often facilitate isolation of the applied electrical field to desired fibers and other neural structures. Specifically, the selection of an electrode 132-135 to function as an anode at a position adjacent to another electrode functioning as a cathode causes the resulting electron/ion flow to be limited to tissues immediately surrounding the two electrodes. By alternating through the possible anode/cathode combinations, it is possible to gain greater resolution in the stimulation of desired tissue or neural structures.

As mentioned above, programmer/controller 140 provides data communication with IPG 110, such as to provide control (e.g., adjust stimulation settings), provide programming (e.g., alter the electrodes to which stimulation pulses are delivered), etc. Accordingly, programmer/controller 140 of the illustrated embodiment includes RF circuitry 141 for establishing a wireless link with IPG 110, and controller 142 to provide control with respect to IPG 110. Programmer/controller 140 may receive data from IPG 110 that can be displayed to medical personnel or a clinician on a screen (not shown) on programmer/controller 140. Additionally, or alternatively, programmer/controller 140 may provide power to IPG 110, such as via RF transmission by RF circuitry 141. Optionally, however, a separate power controller may be provided for charging the power supply 111 within IPG 110.

Additional detail with respect to pulse generation systems and the delivery of stimulation pulses may be found in U.S. Pat. No. 6,609,031, entitled “MULTIPROGRAMMABLE TISSUE STIMULATOR AND METHOD,” the disclosure of which is hereby incorporated herein by reference. Similarly, additional detail with respect to pulse generation systems and the delivery of stimulation pulses may be found in U.S. Pat. No. 7,937,158, entitled “MULTIPROGRAMMABLE TRIAL STIMULATOR.”

Having generally described stimulation system 100 above, the discussion which follows provides detail with respect to various functional aspects of stimulation system 100 according to some embodiments. Although the below embodiments are described with reference to stimulation system 100, and IPG 110 thereof, it should be appreciated that the inventive concepts described herein are not limited to application to the exemplary system and may be used in a wide variety of medical devices.

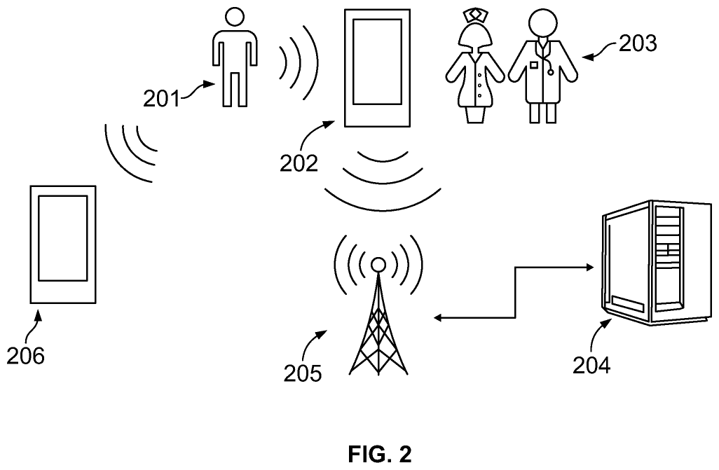

FIG. 2 depicts a system in which an implanted medical device may be programmed and/or monitored by a programmer device to provide SCS or DBS according to some representative embodiments. The implanted medical device (not shown in FIG. 2) is implanted within patient 201. Examples of suitable implantable medical devices include but are not limited to neurostimulators such as the Protege™, Prodigy™, Proclaim™, Infinity™ pulse generators available from Abbott (Plano, TX).

At appropriate times, the implanted medical device of patient 201 communicates with clinician programmer device 202, which is operated by one or more clinicians 203. The programming clinician 203 utilizes one or more user interface screens of device 202 to define or control a therapy provided to patient 201 by the implanted medical device, The clinician(s) may define or set one or more therapy parameters. For example, the clinician may define pulse amplitudes, pulse frequencies, pulse patterns, and/or a variety of other therapy parameters depending upon the implanted device and the intended therapy for patient 201.

During a programming session, programming data may be communicated from clinician programmer device 202 to one or more remote device management servers 204 via network 205. The set of programming data is subjected to authorization and validation processes to ensure that only programming data from authorized clinicians will be accepted by the implanted medical device of patient 201. Suitable security algorithms may be employed to validate and authorize communication between clinician programmer device 202 and servers 204, such as communication of user/clinician identifiers, passwords, device identifiers, network identifiers, security/cryptographic keys, digital certificates, location data, and/or the like. The implanted medical device of patient 201 may also provide information, such as battery life data, to clinician programmer device 202.

In some embodiments, a patient controller 206 can be downloaded onto a patient's smartphone, computer, and the like, and one or more user interface screens can be utilized to facilitate communications with the IPG 110 implanted in the patient 201. The patient controller 206 can turn stimulation therapy ON and OFF, and can adjust one or more therapy settings of the IPG 110 as allowed. For example, the patient controller 206 can communicate an increase and/or decrease of a strength of the stimulation therapy (e.g., effecting an increase/decrease of the voltage, current, and/or charge density of the applied pulses).

In some embodiments, a selected or programmed strength of the delivered therapy pulses can result in a maximum output voltage (VMO) indication, also referred to herein as “compliance voltage”. During VMO, the programmed stimulation parameters demand more voltage than the IPG 110 can provide, such as to sustain an amplitude (e.g., indicative of a level of current, indicative of a level of voltage, etc.) for an entire pulse duration.

When VMO is not occurring, the delivered pulse is flat across the top of the pulse for the entire duration when viewed across a purely resistive load. It should be understood that when the neurostimulation system 100 delivers the pulse in a patient (e.g., not a purely resistive load), the delivered pulse can be substantially flat for the entire duration of the pulse while experiencing some amplitude variation, within acceptable limits, over the pulse duration. In some embodiments, if the pulse cannot be delivered for substantially the entire duration of the pulse, “clipping” or a decrease in the pulse amplitude may occur toward a latter portion of the pulse, or a trailing edge. In this example, if VMO is not occurring but clipping is occurring, the IPG 110 can increase the output of the voltage multiplier 151.

In previous systems, if clipping and VMO occur for the same pulse(s), the amplitude is decreased until the VMO condition is cleared. However, even though the applied therapy is effective at the lower amplitude, this indication can, in some cases, cause unnecessary alarm in the patient 201. In other cases, in therapies where higher level voltage is used, automatically reducing the amplitude when VMO occurs may negatively impact the therapy efficacy.

Therefore, one factor that determines when a current-controlled technology can no longer deliver the full amplitude (e.g., desired amplitude level for the entire pulse duration) using the selected electrode that may have high impedance due to small-size area, is the compliance voltage, or maximum output voltage of the IPG 110. The compliance voltage is typically a multiple of the battery voltage (VBAT). For example, the compliance voltage can be 3.5*VBAT, 3.75*VBAT, 4*VBAT etc.

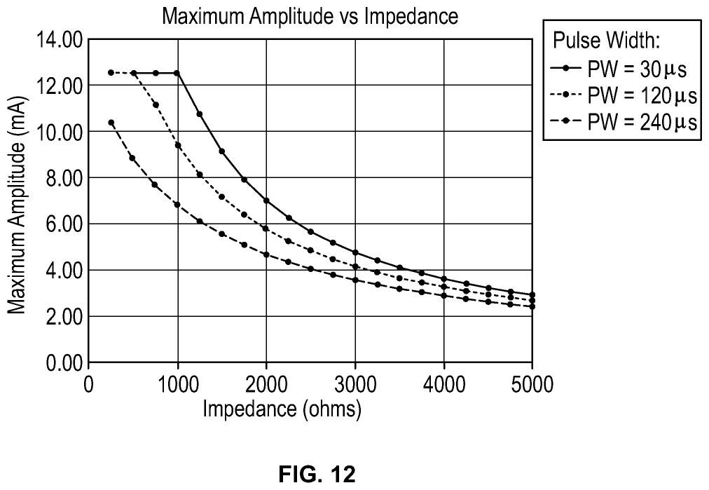

In general, as shown in FIG. 12, as impedance increases, such as when using a single small electrode 131-135, higher amplitudes can be sustained for relatively shorter pulse durations than lower amplitudes. Accordingly, maximum amplitude has a strong dependence on pulse width due to small electrode surface area. FIG. 12 shows the trend lines for example pulse widths at different current amplitudes (mA) for an impedance of an electrode 131-135 in accordance with embodiments herein. By way of example only, for impedance levels up to approximately 1000 ohms, pulses having amplitudes up to approximately 12.5 milliamps (mA) and pulse widths of 30 microseconds (μs) can be sustained at the desired amplitude level. However, as impedance increases beyond approximately 1000 ohms, amplitudes at relatively higher levels may begin to clip, delivering a lower amplitude level for the remainder of the pulse duration. In some cases, for pulses having amplitudes up to approximately 12.5 mA and pulse widths of 120 μs, as impedance increases beyond approximately 500 ohms, the amplitude may begin to clip, delivering a lower amplitude level for the remainder of the pulse duration. In other cases, for pulses having amplitudes up to approximately 10.25 mA and pulse widths of 240 μs, as impedance increases beyond approximately 250 ohms, the amplitude may begin to clip, delivering a lower amplitude level for the remainder of the pulse duration.

Accordingly, the maximum amplitude indicative of a level of current has a strong dependence on pulse width due to the small electrode surface area when using single electrodes 131-135 to deliver therapy. While many directional therapies, such as therapies for PD which can be below 3.2 mA and 90 μs, do not experience clipping, therapies, such as for Dystonia that use higher amplitude levels may experience clipping that may currently be indicated by an error message and/or an auto-reduction in amplitude. This may result in concern on the side of the patient and/or clinician, even though the efficacy of the therapy may not be negatively impacted.

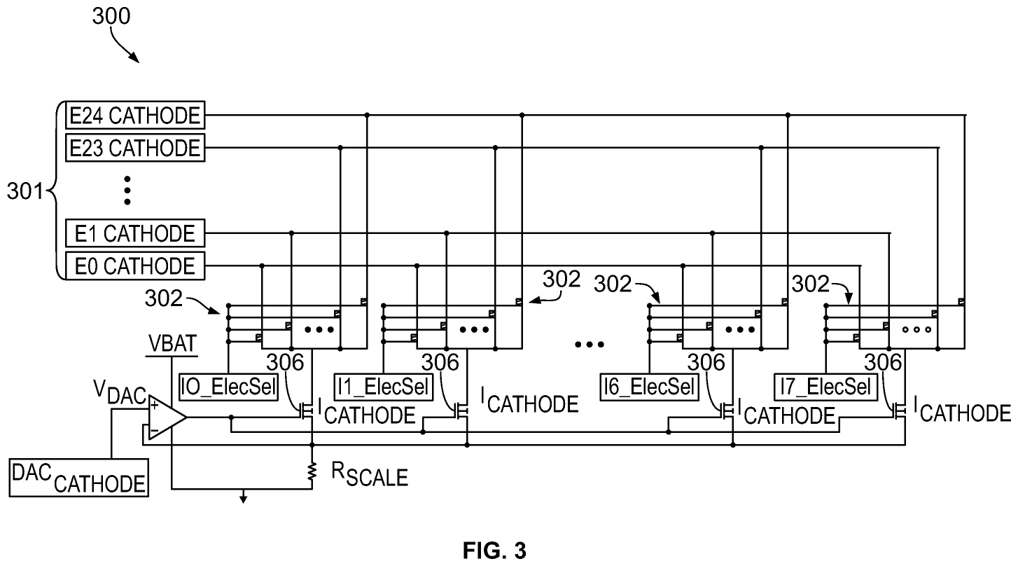

FIG. 3 depicts a cathode-side programmable amplitude current regulator 300 that can be used in some embodiments. In some cases, the cathode-side programmable amplitude current regulator 300 can be utilized for providing active discharge pulses in an IPG as described in additional detail in U.S. Patent Publication Number 2021/0402192 entitled “IMPLANTABLE PULSE GENERATOR FOR PROVIDING A NEUROSTIMULATION THERAPY USING COMPLEX IMPEDANCE MEASUREMENTS AND METHODS OF OPERATION” published Dec. 30, 2021, which is hereby incorporated herein by reference in its entirety.

The programmable amplitude current regulator 300 comprises a plurality of cathode electrodes 301 that may be programmed as the cathode from the patient. An IPG delivers stimulation in an embodiment by generating an anode voltage with a voltage multiplier (not shown). The anode voltage is connected to an anode electrode that is implanted in the patient. The IPG also programs one or more cathode electrodes 301 that pull the current from the anode electrode. The voltage originates from the voltage multiplier, flows out of the anode electrode, through the patient to the cathode electrode, and then back through the cathode electrode through current regulator 300. Electrode selection circuits 302 are used to select which cathode electrode 301 is active. In an embodiment, electrode selection circuits 302 use field-effect transistor (FET) transistors for electrode connectivity to the current regulator.

Digital to analog converter (DAC) cathode 303 is a digitally-controlled voltage source that provides a reference voltage. DAC cathode 303 provides an input voltage (V_{DAC}) to an error amplifier 304, The voltage for the other input to error amplifier 304 is set by programmable resistor (R_{SCALE}) 305, which may be, for example, a resistance network that is programmed digitally. The stimulation therapy current (I_{CATHODE}) that is provided to the patient is determined by Ohm's law: I=V_{DAC}/R_{SCALE}. This current is programmed to last for a certain duration that is referred to herein as pulse width or pulse duration. The cathode current is output for a limited duration and is then shut off. In some embodiments, when VMO does not occur, the cathode current essentially provides a constant current pulse for a given amount of pulse width. In other embodiments, when VMO occurs, the level of the amplitude of the current pulse may decrease, as further discussed herein.

In an embodiment such as IPG 110 (FIG. 1), electrode selection circuits 302 and connections to cathode electrodes 301 may be components of lead connectors 153 and current generator elements (DAC 303, error amplifier 304, and variable resistor 305) may be components of voltage/current control 152. Electrodes 132-135 on lead body 131 may function as anodes or cathodes.

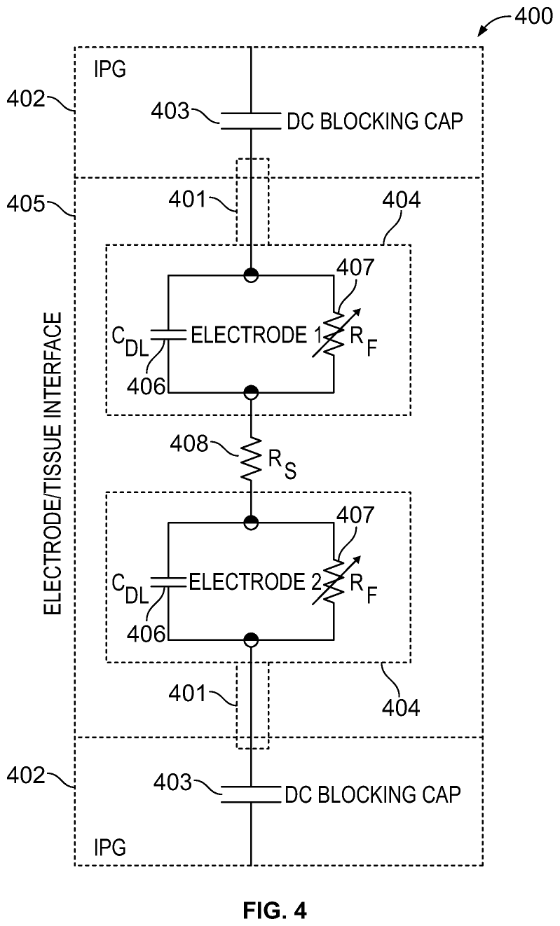

FIG. 4 depicts a model 400 of the electrical interaction between a patient's tissue and stimulation electrodes. One or more stimulation leads 401 are connected to an IPG 402. The stimulation leads 401 are coupled to IPG 402 via DC blocking capacitors 403, which for safety reasons prevent DC current from being applied to the patient's tissue. As a component of IPG 402, the DC blocking capacitors 403 have a known value. In an example embodiment, DC blocking capacitors are 22 μF. Stimulation leads 401 comprise one or more electrodes 404, such as electrodes 132-135 (FIG. 1). When a stimulation therapy waveform is applied to a patient, one electrode 404 may be designated as an anode and the other electrode 404 is designated as a cathode such that charge flows from the anode electrode 404 to the cathode electrode 404. In an embodiment, one electrode may be the metallic case of IPG 402 that functions as an anode.

Model 400 depicts an electrode/tissue interface 405 that represents the electrical characteristics of the physical electrodes that reside in the patient's tissue. The electrode/tissue interface 405 can be modeled as an RC network for each electrode comprising a parallel capacitor (C_{DL}) 406 in parallel with a variable resistance (R_{F}) 407, which are then in series with at least some portion of the patient tissue resistance (R_{S}) 408. Capacitor 406 represents the capacitive interface between the electrode and the tissue, which is a double layer (DL) capacitance. At the interface where the physical electrode 404 touches the patient's tissue, electrode 404 acts like a capacitor, which is represented by capacitor (C_{DL}) 406. Where the electrode 404 touches the patient's tissue there are also resistances, which is represented by Faradaic resistance (R_{F}) 407 and some portion of the patient tissue resistance (R_{S}) 408. Variable resistance 407 accounts for Faradaic conduction across the electrode/tissue interface that is dependent upon the stimulation current density. Resistance (R_{S}) 408 represents the resistance through and across the patient's tissue between electrodes 404, which is dependent upon the conductivity of the tissue and the effective surface areas of the electrodes. It will be understood that the specific values assigned to the resistances and capacitors in model 400 are dependent upon the type and deployment of electrodes in a particular patient and, therefore, model 400 is unique for each patient and stimulation lead.

The model 400 of an actual electrode/tissue interface 405 in a patient can be determined by taking voltage measurement samples, such as can be taken by IPG 402 across stimulation leads 401 and/or electrodes 404. If only a single voltage measurement is taken, then the electrode/tissue interface can be modelled only as a single resistance, which may or may not be adequate in assessing the impact of the electrodes upon the stimulation delivered by the IPG. However, this is an over-simplification of the load into which the IPG device delivers stimulation. IPG stimulation therapy is not instantaneous, but instead occurs over a period of time, Accordingly, model 400 represents a complex impedance load network for the IPG, which can take time-dependent effects into account, such as caused by capacitances in a load network. By determining model 400 for a patient, IPG 402 can monitor shifts in resistance and capacitance (e.g., R_{F}, C_{DL}, or R_{S}) over time during the life of the patient and device. This provides improved monitoring of lead integrity and patient health and position in some cases.

In an example embodiment, the following parameters may be used to model a directional DBS stimulation electrode:
Effective Electrode/Patient Interface Capacitance (C_{DL})=0.1 μF;
Patient Load Resistance (R_{S})=2KΩ; and
Residual Voltage after Stimulation (V_{RESIDUAL})=3.6V (3 mA, 120 μS).

The Initial Discharge Amplitude (I_{INITIAL}) can be calculated as:

I_{INITIAL}=V_{RESIDUAL}/R_{LOAD}=1.8 mA

The Discharge Time Constant (T) can be calculated as:

τ=R_{S}*C_{DL}=0.2 mS.

### Current Regulator Circuit

FIG. 5 depicts a current regulator circuit 500 configured to output a current-controlled output and an amplitude monitoring signal 502 (IERROR, by way of example) in accordance with embodiments herein. In this embodiment, current is controlled by an active current regulator with fine (Amplitude Control 504) and coarse control (SCALE control 506). The current regulator circuit 500 can be incorporated within the voltage/current control 152 discussed in FIG. 1, and may be integrated with portions or all of FIG. 3, such as integrate with other functionality as well as be interconnected with the lead connectors 153.

The amplitude monitoring signal 502 (IERROR) can output a status or result based on a comparison of a present amplitude level to one or more threshold, or can simply detect clipping of the delivered current-controlled pulse. For example, if the amplitude level output by the current regulator circuit 500 is equal to or greater than the one or more threshold, a first status can be output on the signal 502, and if the amplitude level output by the current regulator circuit 500 is less than the one or more threshold, a second status can be output on the signal 502. In some embodiments the signal 502 can output a first level (e.g., 0 volts or other level) for a first status and a second level (e.g., 5 volts or other level) for a second status.

As discussed further below, the signal 502 can be monitored for at least a sub-duration of the entire pulse duration. The signal 502 can be used to indicate that i) the selected/programmed current amplitude (e.g., initial criteria) is achieved for the sub-duration of the entire pulse duration (e.g., the pulse is valid) or ii) the selected/programmed current amplitude is not achieved for the sub-duration of the entire pulse duration (e.g., the pulse is invalid). If the current amplitude is not achieved for the sub-duration and the voltage level being supplied by the voltage multiplier 151 is not at VMO, the voltage multiplier 151 can be directed to increase the output voltage. If the current amplitude is not achieved for the sub-duration and the voltage level being supplied by the voltage multiplier 151 is at VMO, if a voltage auto-reduce feature is inhibited, the voltage level being supplied by the voltage multiplier 151 is maintained. If the current amplitude is not achieved during a time period following the sub-duration, the IPG 110 can maintain the voltage level supplied by the voltage multiplier 151 and the amplitude level is allowed to decrease or clip along the trailing edge of the pulse.

Although not shown, a voltage monitoring circuit can be used to measure a voltage signal that is complementary to the monitoring signal 502. Accordingly, either a voltage amplitude level or a current amplitude level can be used to determine whether the pulse amplitude maintains the selected amplitude for the sub-duration.

### Monitoring Amplitude During Sub-Duration of Pulse Duration

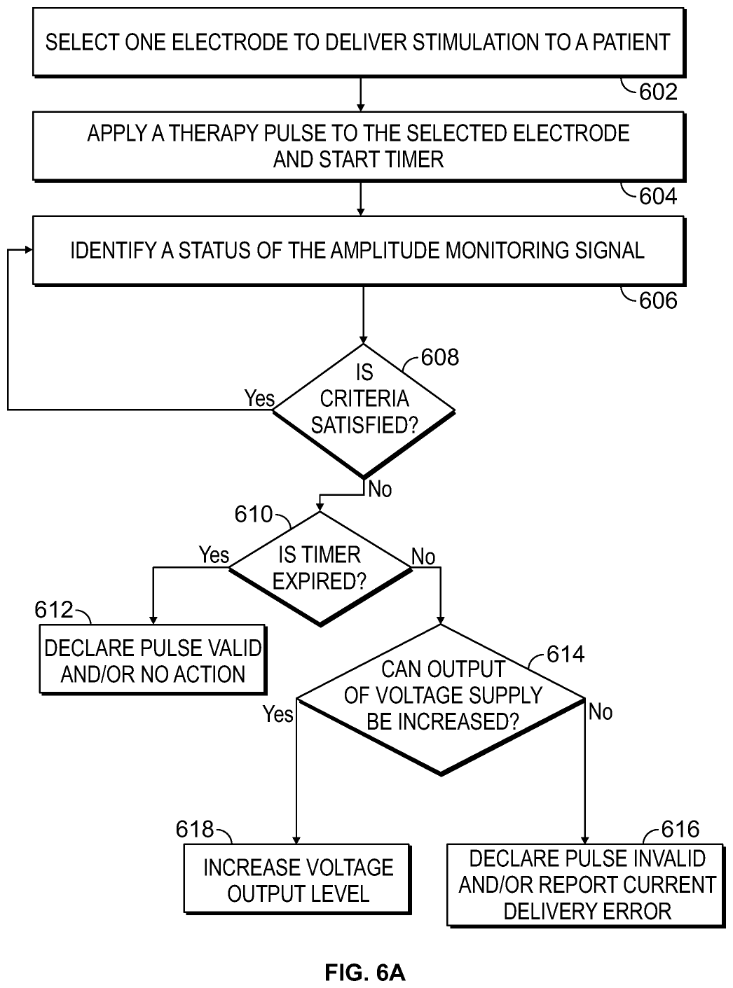

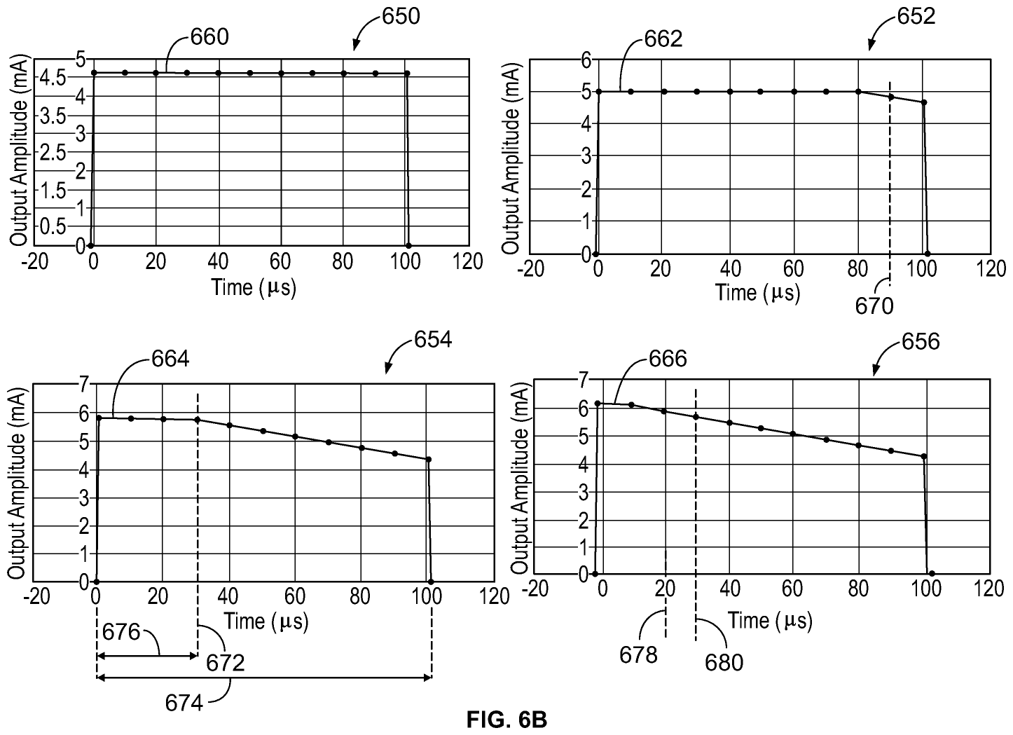

FIG. 6A illustrates a method for delivering and monitoring pulses to ensure that a sub-duration of the entire pulse duration maintains a desired amplitude in accordance with embodiments herein. The operations of FIG. 6A may be implemented by hardware, firmware, circuitry and/or one or more processors housed partially and/or entirely within an IPG 110, a local external device (e.g., programmer device 202, patient controller 206, etc.), remote server or more generally within a health care system. Optionally, the operations of FIG. 6A may be partially implemented by an IPG 110 and partially implemented by a local external device, remote server or more generally within a health care system. For example, the IPG includes IPG memory and one or more IPG processors, while each of the external devices/systems (e.g., local, remote or anywhere within the health care system) include external device memory and one or more external device processors.

At 602, one or more processors select one or more electrodes 132-135 to deliver stimulation to a patient using the neurostimulation system 100. In some embodiments, for targeted therapy, a single electrode 132-135 is selected. At 604, the one or more processors apply a therapy pulse from the IPG 110 to the selected electrode 132-135 and start a timer. In some embodiments, a timer is not used, but the timer is discussed herein for clarity of the method. For example, the timer can be associated with a number of clock cycles, such as the first N clock cycles and/or a predetermined length of time (e.g., a sub-duration) that is less that the entire pulse duration of the pulse. Therefore, a “timer” can also include a counter that counts a number of clock cycles or monitors the length of time of the sub-duration. In at least some embodiments, the sub-duration can be started at the beginning (e.g., leading edge of the pulse) or very near the leading edge of the pulse.

At 606, the one or more processors identify a status of the amplitude monitoring signal 502, such as at every clock cycle, every M clock cycles, every predetermined time duration such as every μs, every five μs, and the like.

At 608, the one or more processors determine whether the status of the amplitude monitoring signal 502 indicates that a present level of the amplitude of the pulse satisfies a criteria. In some embodiments the criteria can be an initial criteria, while in others the criteria may be modified by adjusting the input voltage level from the voltage multiplier 151, which may or may not modify the initial criteria. In some embodiments, the initial criteria can include one or more threshold level such as an amplitude or amplitude range that is indicative of a level of current, while in other embodiments, the initial criteria can include an amplitude or amplitude range that is indicative of a level of voltage. In some cases, the range can be defined by a narrow band of values, allowing some acceptable fluctuations in the amplitude level of the signal.

If the criteria are satisfied, the process can return to 606. In some embodiments the sub-duration/timer may be evaluated, and if the number of clock cycles/predetermined time duration has been met (e.g., the sub-duration has been exceeded in time), the one or more processors may stop determining the status at 606 until the next therapy pulse starts at 604.

If the criteria are not satisfied at 608, such as if the amplitude of the pulse is less than the one or more threshold level, the process flows the 610 and the one or more processors determine whether the sub-duration/timer is expired. If the timer is expired, the sub-duration of the pulse duration has passed. Flow passes to 612 and the one or more processors declare the pulse valid and/or no action is taken, because the amplitude level of the pulse remained at the desired threshold level for the sub-duration of the pulse duration. Further, no error message is set and the output of the voltage multiplier 151 is not adjusted. Because the first part of the pulse, the sub-duration, is at the programmed/selected amplitude, which in some embodiments can be a relatively higher amplitude, the effect on charge delivered is limited. That is, the charge may only be slightly decreased in comparison with decreasing the output of the voltage multiplier 151.

Returning to 610, if the timer is not expired, the pulse is still being delivered within the sub-duration. This condition indicates that the amplitude of the pulse is less than the at least one threshold level for at least a portion of the sub-duration of the entire pulse duration. The method passes to 614 and the one or more processors determine whether the output of the voltage multiplier 151 can be increased. In some embodiments, if the voltage multiplier 151 is delivering a voltage that is less than, for example, the top one or two voltage multiplier settings, the one or more processors can determine that the voltage is not at a maximum level and can be increased. In other embodiments, the one or more processors can determine whether the voltage is equal to or greater than one or more voltage threshold value.

If the voltage cannot be increased, at 616 the one or more processors declare the pulse invalid and/or report a current delivery error. In some embodiments, this can indicate that the current regulator circuit 500 is unable, under the selected settings, to deliver the selected amplitude (e.g., current, voltage, etc.) for the sub-duration.

Returning to 614, if the one or more processors determine that the output of the voltage multiplier 151 can be increased, the method passes to 618 and the one or more processors direct the voltage multiplier 151 to increase the output voltage level. In some embodiments, the voltage level can be increased one increment or step. It is advantageous to use the lowest setting to supply the desired pulse to conserve battery power.

In some embodiments, a plurality of pulses can be delivered. One or more pulses can satisfy the initial criteria (608), such as for the entire pulse duration, and be declared valid pulses. Another pulse can satisfy the initial criteria for the sub-duration, but not for the entire pulse duration, and be declared a valid pulse. Yet another pulse that does not satisfy the initial criteria for the sub-duration is declared an invalid pulse.

FIG. 6B illustrates graphs showing different pulses at different amplitudes in accordance with embodiments herein and will be discussed with respect to the method of FIG. 6A. The horizontal axis illustrates time in μs, while the vertical axis illustrates output amplitude of the pulse in milliamps (mA). Graphs 650, 652, 654, 656 illustrate pulses 660, 662, 664, 666, respectively. Each of the pulses 660-666 has an entire pulse duration of approximately 100 μs, although other pulse durations can be used.

Turning to graph 650, the pulse 660 maintains the amplitude, indicated as approximately 4.6 mA, for the entire pulse duration of 100 μs. Accordingly, the criteria, namely the amplitude of the level of current at 4.6 mA, is satisfied for the entire pulse duration and the status of the amplitude monitoring signal at 606 is not set to indicate that the pulse is invalid (e.g., the pulse 660 is a valid pulse).

Turning to graph 652, the pulse 662 maintains the amplitude, indicated as approximately 5 mA, until approximately 90 μs, indicated with dotted line 670. If the sub-duration of the pulse duration is less than 90 μs, the timer has expired (e.g., FIG. 6A at 610) prior to the amplitude decrease and the pulse is declared valid. In another embodiment, if the timer has expired (e.g., the sub-duration has elapsed) and the status of the amplitude monitoring signal 502 indicates that the amplitude is out of range, the one or more processors ignore the amplitude monitoring signal 502.

In graph 654, the pulse 664 maintains the amplitude, indicated as approximately 5.75 mA, for more than 30 μs. In some embodiments, the sub-duration 676 of the entire pulse duration 674 can be approximately 30 μs, although other sub-durations are contemplated, such as 25 μs, 35 μs, 40 μs, and the like. In this example wherein the sub-duration 676 is indicated by dotted line 672, the criteria is satisfied at 608 of FIG. 6A as the status of the amplitude monitoring signal 502 indicates that the amplitude is in range. The amplitude of the pulse 664 is clipped or begins to decrease after 30 μs. At this point, the amplitude level is controlled by the voltage level provided by the voltage multiplier 151. Because the amplitude was sustained at the desired amplitude level for at least the sub-duration 676, the delivered therapy resulted in the desired effect for the patient. The pulse 664 is declared valid.

Turning to graph 656, the pulse 666 starts with an amplitude of approximately 6.25 mA. The amplitude begins to clip after approximately 10 μs. For example, the amplitude monitoring signal 502 at 20 μs, indicated by dotted line 620, may indicate that the amplitude level is out of range. If the criteria is not satisfied at 608 and the timer is not expired at 610 (e.g., the pulse is within the sub-duration, such as within the first 30 μs as indicated by dotted line 680), the output of the voltage multiplier 151 can be increased if the voltage supply is not at a maximum or predetermined level. In the example of graph 656, the output of the voltage multiplier 151 is already at the maximum level and thus the pulse is declared invalid (at 616).

### Monitoring Amplitude of Pulse and Status of Auto-Reduce Feature

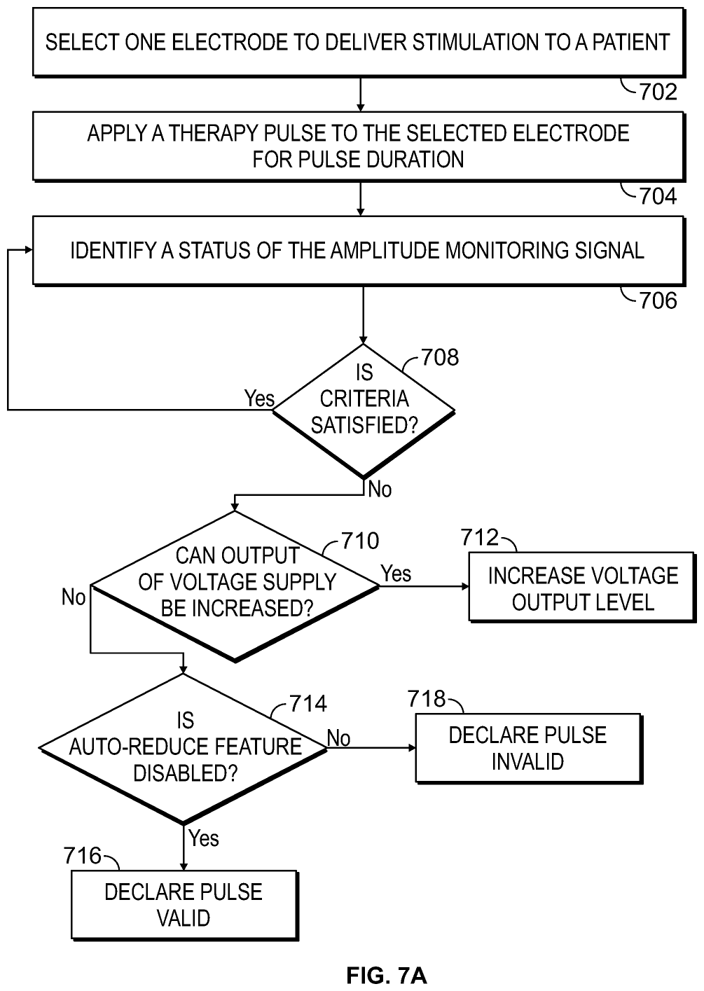

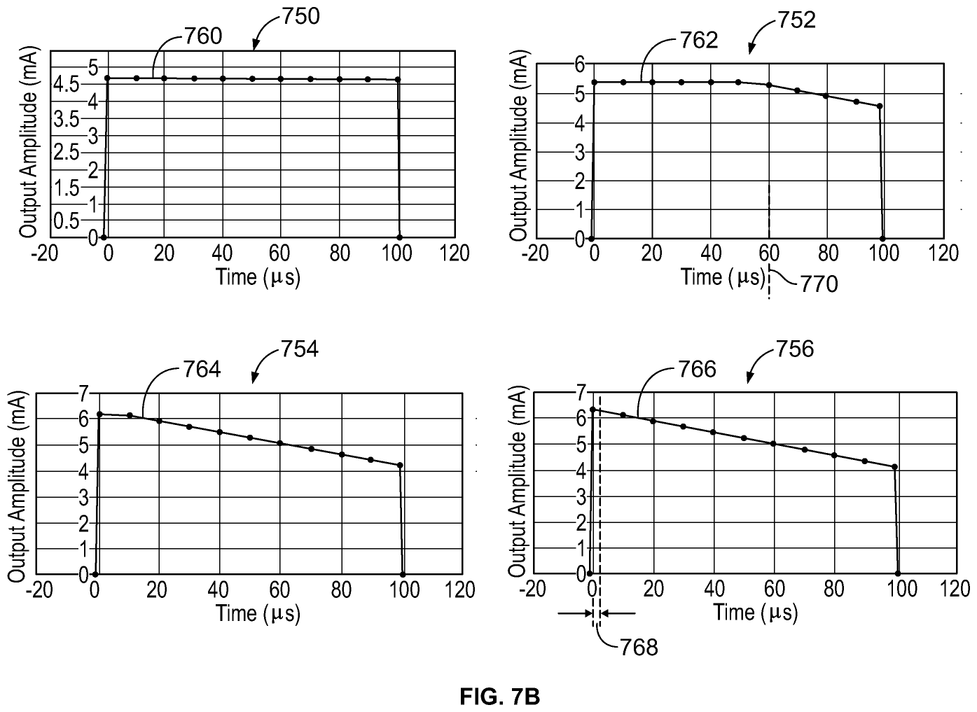

FIG. 7A illustrates a method for delivering and monitoring pulses while allowing the amplitude of the pulse to decrease or clip while maintaining the input voltage from the voltage multiplier 151 in accordance with embodiments herein. The operations of FIG. 7A may be implemented by hardware, firmware, circuitry and/or one or more processors housed partially and/or entirely within an IPG 110, a local external device (e.g., programmer device 202, patient controller 206, etc.), remote server or more generally within a health care system. Optionally, the operations of FIG. 7A may be partially implemented by an IPG 110 and partially implemented by a local external device, remote server or more generally within a health care system. For example, the IPG 110 includes IPG memory and one or more IPG processors, while each of the external devices/systems (e.g., local, remote or anywhere within the health care system) include external device memory and one or more external device processors.

At 702, one or more processors select one or more electrodes 132-135 to deliver stimulation to a patient using the neurostimulation system 100. In some embodiments, for targeted therapy, a single electrode 132-135 is selected. At 704, the one or more processors apply a therapy pulse from the IPG 110 to the selected electrode 132-135.

At 706, the one or more processors identify a status of the amplitude monitoring signal 502, such as at every clock cycle, every M clock cycles, every predetermined time duration such as every μs, every five μs, and the like. In contrast with FIG. 6A, the method of FIG. 7A does not monitor the amplitude monitoring signal 502 for a predetermined sub-duration of the pulse duration; however, the sub-duration of the method of FIG. 7A can be equal to one or more clock cycles, one or more μs, and the like, and thus a very short time duration. In some embodiments, the sub-duration may be the same as the leading edge of the pulse, thus monitoring a level of the signal just at the beginning of the pulse duration.

At 708, the one or more processors determine whether the status of the amplitude monitoring signal 502 indicates that a present level of the amplitude of the pulse satisfies the initial criteria as discussed in FIG. 6A. If the criteria are satisfied, the process can return to 706.

If the criteria are not satisfied at 708, the process flows to 710 and the one or more processors determine whether the output of the voltage multiplier 151 can be increased. In some embodiments, if the voltage multiplier 151 is delivering a voltage that is less than the top one or two voltage multiplier settings, the one or more processors can determine that the voltage is not at a maximum level and can be increased. In other embodiments, the one or more processors can determine whether the voltage is equal to or greater than one or more voltage threshold value to determine whether the voltage can be increased. For example, if the voltage multiplier 151 is set at a top setting, such as the top one or two settings, the voltage multiplier 151 is already providing the maximum level of voltage available.

If the voltage can be increased, the method passes to 712 and the one or more processors increase the voltage output level of the voltage multiplier 151. In some embodiments, the amplitude of the voltage and/or amplitude of the current can be increased based on input from the patient controller 206.

Returning to 710, if the output of the voltage multiplier 151 cannot be increased, VMO (e.g., maximum output voltage, compliance voltage, etc.) is detected, and the method passes to 714. In some embodiments, at 714 the one or more processors can determine whether the auto-reduce feature 122 is disabled. For example, the auto-reduce feature can be an indication to reduce the voltage output from the voltage multiplier 151, such as, to keep the amplitude of the pulse constant for the pulse duration. In some embodiments, the clinician programmer device 202 can pass a flag or other indication to the IPG 110 to disable the auto-reduce feature 122 of the patient controller 206 in predetermined situations, such as with identified therapies, at predetermined voltage levels, etc. Disabling the auto-reduce feature 122 provides an indication that the voltage level is not to be reduced. If the auto-reduce feature 122 is disabled, the method passes to 716 where the one or more processors can declare the pulse valid, maintain the voltage level used to generate the pulses (e.g., output of the voltage multiplier 151 is not adjusted), and pulses can continue to be administered even when VMO is detected. Also, in some embodiments no VMO error (e.g., error message) is output to the clinician programmer 202 or the patient controller 206.

At 714, if the auto-reduce feature 122 is not disabled, the method passes to 718 and, in some cases, the pulse can be declared invalid and/or trigger the VMO condition. In some embodiments, the IPG 110 will facilitate the auto-reduce feature 122 to reduce the voltage output from the voltage multiplier 151.

FIG. 7B illustrates graphs showing different pulses having different amplitudes in accordance with embodiments herein and will be discussed with respect to the method of FIG. 7A. The horizontal axis illustrates time in μs, while the vertical axis illustrates output amplitude of the pulse in mA. Graphs 750, 752, 754, 756 illustrate pulses 760, 762, 764, 766, respectively. Each of the pulses 760-766 has an entire pulse duration of approximately 100 μs, although other pulse durations can be used. The sub-duration can be shorter than in the method of FIG. 6A; namely, in some embodiments the sub-duration can be a single amplitude measurement or several consecutive amplitude measurements sampled at or proximate the beginning of the leading edge of the pulse.

Turning to graph 750, the pulse 760 maintains the amplitude, indicated as approximately 4.6 mA, for the entire pulse duration. In some embodiments, if VMO is not detected at 710 (FIG. 7A), the therapy pulse 760 is delivered without an error and can be declared a valid pulse. However, if VMO is detected at 710 and the auto-reduce feature is disabled at 714, the IPG 110 continues to deliver the therapy without modifying setting. In the example of the pulse 760, the amplitude of the current and the output of the voltage multiplier 151 are maintained even if VMO is detected.

In graph 752, the pulse 762 maintains the amplitude, indicated as approximately 5.5 mA, until approximately 60 μs, indicated with dotted line 770. If VMO is detected at 710 at or after time at line 770, and the voltage multiplier is at the highest allowed level, the voltage multiplier 151 will maintain the output voltage level, allowing the amplitude of the pulse 762 to be clipped and/or decrease during the trailing portion of the pulse 762 if the auto-reduce feature is disabled.

In graph 754, the pulse 764 maintains the amplitude, indicated as approximately 6.25 mA, for approximately 10 μs. As with the pulses 760 and 762, if VMO is detected at 710 and the auto-reduce feature is disabled, the voltage multiplier 151 maintains the output voltage level while the amplitude of the pulse 764 decreases during the trailing portion.

Turning to graph 756, the pulse 766 starts with an amplitude of approximately 6.5 mA. The amplitude begins to clip soon after the leading edge or beginning of the pulse 766. In this example, sub-duration 768 can be as short as several μs. Accordingly, the criteria of the amplitude level and the sub-duration are satisfied. The therapy can continue without changes to the setting(s) and without indicating an error if the auto-reduce feature is disabled.

In accordance with new and unique aspects, the IPG 110 delivers a particular treatment for the medical treatment of neurological conditions such as, but not limited to, Parkinson's Disease and Dystonia. The treatment of the neurological condition includes applying pulses that have a pulse duration and amplitude to at least one element 131-135. The signal can be monitored to ensure that the programmed/desired amplitude is administered to the patient for the sub-duration of the pulse. Accordingly, the output range can be expanded and the charge delivered to the patient remains in a therapeutic range.

Further, the IPG 110 delivers the particular treatment which transforms the nervous system of a patient to a normal activity state, wherein the nervous system can cause abnormal or undesirable movements/activity in the patient prior to treatment. The processor 115 can determine whether the therapeutic pulse meets the initial criteria, can increase the output of the voltage multiplier 151 if the multiplier is not at or near a maximum setting, and can determine if the auto-reduce feature is disabled. If the auto-reduce feature is disabled, the trailed edge of the therapeutic pulse can be clipped while continuing the therapy and treatment of the patient's condition.

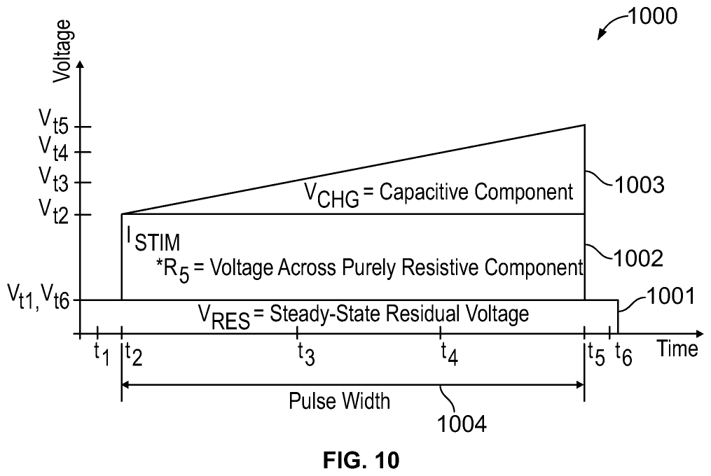

Embodiments of blending current and voltage control for neuromodulation output provide the following and other advantages by delivering the full output current for at least a portion (e.g., sub-duration) of the pulse duration:
optimize output range and efficiency for activating a single segment of a directional lead,
expand the available output range of the device while still ensuring that the delivered charge is increasing with an increase in amplitude or pulse width, as shown below in FIG. 10,
improve battery current efficiency at settings that are not near the maximum output by allowing the voltage multiplier 151 to run one to two steps lower than the maximum, and/or at lower settings,
use an operational amplifier-based current regulator (e.g., FIG. 5) that can detect an output at the supply rail,
maintain patient safety as charge density and total charge delivered at the programmed current strength are still in control and only reduced, not increased,
preserve monotonicity where an UP command requesting an increase in controller amplitude corresponds to an increase in charge delivered,
when used at high settings, the effect on charge delivered is negligible compared to a purely current-controlled pulse, and
deliver higher amplitudes into higher impedances to cover a wider range of neurological targets and therapies.

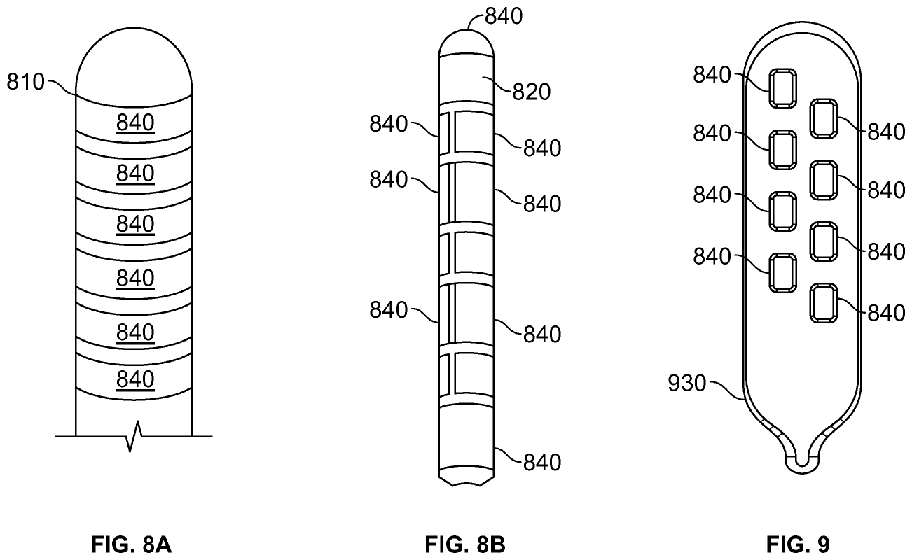

FIGS. 8A-8B respectively depict stimulation portions 810, 820 for inclusion at the distal end of a stimulation lead 130 according to example embodiments. FIG. 9 depicts paddle lead stimulation portion 930 according to example embodiments. Stimulation portions 810, 820, and 930 each include one or more electrodes 840. Stimulation portion 810 depicts a conventional stimulation portion of a “percutaneous” lead with multiple ring electrodes. Stimulation portion 820 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. Pat. No. 9,054,436, entitled, “METHOD OF FABRICATING STIMULATION LEAD FOR APPLYING ELECTRICAL STIMULATION TO TISSUE OF A PATIENT,” which is incorporated herein by reference. Stimulation portion 930 includes multiple planar electrodes on a paddle structure.

As discussed further below in FIG. 10, embodiments are directed to a method for using voltage waveform capture of a current-controlled therapeutic stimulation waveform to provide modelling of a complex impedance for the electrode/tissue interface. Instead of requiring external test equipment, this measurement capability may be included in an implantable device, which allows for long-term patient monitoring and more realistic modelling of tissue load. Most accepted electrode/tissue interface models are represented by a complex impedance network having capacitors and variable resistors in addition to simple resistances, so multiple samples over time become necessary to accurately determine the load model for the stimulation device.

FIG. 10 is a plot 1000 illustrating the components of the voltage across load model 400 (FIG. 4) resulting from a constant-current therapy pulse. Voltage component 1001 is the steady-state residual voltage (VRES) that is present on stimulation leads 401 and/or electrodes 404. This voltage may correspond to the undischarged voltage that has built up during prior therapy pulses and, therefore, may exist before and after the therapy pulse is generated. Voltage component 1002 corresponds to the voltage created by the stimulation current (ISTIM) across the resistive component R_{S} 408 of the electrode/tissue interface. Component 1003 corresponds to the voltage (VCHG) added by charge on capacitors CDL 406, which increases throughout the duration 1004 of the therapy pulse. Knowledge of the double-layer capacitance value is a critical input to understanding the electrode/tissue interface. It is known that both the double-layer capacitance CDL 406 and the Faradaic conduction RF 407 vary with stimulation current density. As a result, it is important to be able to take complex impedance measurements using the delivered therapeutic stimulation pulse.

In accordance with embodiments herein that provide blended current and voltage control for the neuromodulation output, the available output range is expanded while ensuring that the delivered charge is increasing with an increase in amplitude or pulse width. In some embodiments the slope increases for relatively smaller electrodes.

Further, electrode geometries have a direct impact on the impedance a stimulation system must overcome to deliver a current-controlled pulse. Smaller electrodes increase impedance overall and put strains on systems that are trying to ensure 100% of the current is delivered to the end of the pulse. This is due to the increase in rate of charging the small capacitance of a small-geometry electrode, such as a segmented lead for DBS.

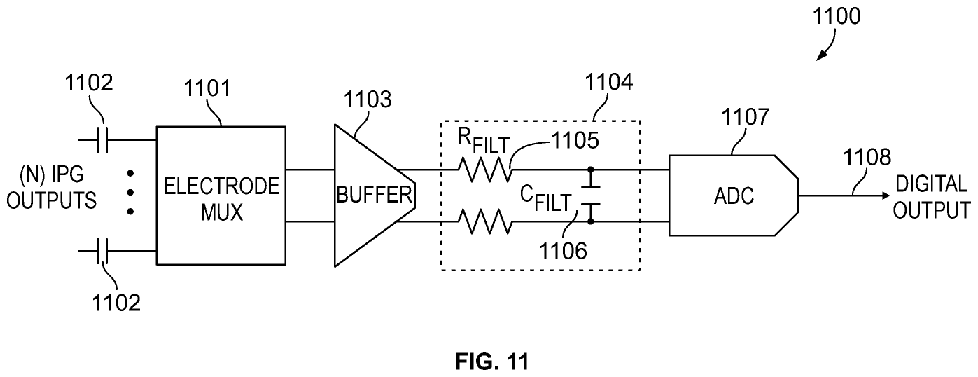

FIG. 11 depicts a circuit 1100 that can be used for the complex impedance measurements of an electrode/tissue interface. Electrode multiplexer (MUX) 1101 is coupled to a plurality (N) IPG electrode leads (not shown). Electrode MUX 1101 may be connected to the electrodes through stimulation or sensing DC blocking capacitors 1102. Electrode MUX 1101 selects sets of the IPG electrode leads to be evaluated and sends signals from those leads to buffer 1103. Differential buffer 1103 attenuates the voltage signal of the load so that measurements can be made using therapeutic stimulation waveforms. Electrode MUX 1101 selects sets of the IPG electrode leads to be evaluated and sends buffered voltage signals from those leads to filter circuit 1104, comprising the two resistances RFILT 1105 and CFILT 1106, isolates analog-to-digital converter (ADC) 1107 and prevents aliasing. The differential ADC 1107 digitizes signals of either positive or negative polarity and outputs digital data 1108. Circuit 1100 captures entire stimulation response voltage waveforms from the electrode leads. Alternatively, circuit 1100, may capture strategic measurement points on each load response waveform at multiple times for simpler post-processing calculations in generating the complex impedance model of the electrode/tissue interface. The captured waveforms and/or measurement points on each waveform represented by output 508 may be stored in memory for additional processing to evaluate the waveform components.

Referring to FIG. 10, for example, circuit 1100 may be used to capture voltages for a stimulation therapy pulse at different time points t1 through t6, which occur before, during, and after the stimulation therapy is applied. The voltage measured at time points t1 and t6 occur outside the delivery of therapy. Accordingly, the voltages (VRES) at these intervals are associated with residual voltage on the electrodes. Once identified, this voltage can be subtracted from measurements captured during the stimulation therapy. The voltages measured at time points t2 and t5 occur at the beginning and end of the stimulation therapy, respectively. The stimulation therapy is typically applied at a constant current ISTIM for a certain time duration or pulse width. Using this known current and the measured voltage at the start of the therapy pulse (i.e., at time t2), the value of the patient's tissue resistance (RS 408, FIG. 4) can be calculated using Ohm's law (e.g., RS=Vt2−Vt1/ISTIM).

As the current from the stimulation therapy is applied to the electrodes, charge builds up on the double layer capacitor (CDL 406, FIG. 4) in the electrode/tissue interface model. The value of the double layer capacitor can be determined by measuring the change in voltage as charge is delivered (i.e., C=Q/ΔV where C is the value of the capacitor, Q is the charge on the capacitor, and ΔV is change in voltage across the capacitor). The value of ΔV can be determined from the slope of segment 1003 in FIG. 10, such as the difference between Vt2 and Vt5 when a constant-current stimulation therapy pulse starts and stops or from the difference between other voltages during the therapy pulse, such as between Vt3 and Vt1. The amount of charge Q can be estimated from the integration of the therapy current I_{STIM} over the pulse width 1004 duration. Using these values, the double layer capacitor (CDL) value can be estimated. The value of the Faradaic resistance (R=407, FIG. 4) in the electrode/tissue interface model may be estimated by nonlinearities in the measured voltage versus time, based upon the estimated CDL capacitance and stimulation current density, for example.

The electrode/tissue interface may be modeled continuously (i.e., for each therapy pulse) or intermittently (e.g., at predetermined intervals, or upon the occurrence of events, such as changes in the therapy schedule). Models of the electrode/tissue interface may be calculated for each set of anode/cathode electrodes that are used to provide therapy and/or that are implanted in the patient. These models may be compared over time (and/or at various stimulation current densities) to evaluate the operation of the electrodes, leads, and/or IPG and to detect changes in the device and/or the patient's health. As discussed herein, the IPG may modify its emulated passive discharge according to a most recent electrode/tissue model calculated from the measurements.

One or more of the operations described above in connection with the methods may be performed using one or more processors. The different devices in the systems described herein may represent one or more processors, and two or more of these devices may include at least one of the same processors. In one embodiment, the operations described herein may represent actions performed when one or more processors (e.g., of the devices described herein) execute program instructions stored in memory (for example, software stored on a tangible and non-transitory computer readable storage medium, such as a computer hard drive, ROM, RAM, or the like).

The processor(s) may execute a set of instructions that are elements may also store data or other information as desired or needed. The storage element may be in the form of an information source or a physical memory element within the controllers and the controller device. The set of instructions may include various commands that instruct the controllers and the controller device to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

The controller may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. When processor-based, the controller executes program instructions stored in memory to perform the corresponding operations. Additionally, or alternatively, the controllers and the controller device may represent circuits that may be implemented as hardware. The above examples are exemplary only and are thus not intended to limit in any way the definition and/or meaning of the term “controller”.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

1. An implantable pulse generator (IPG), comprising:
circuitry configured to control generation and delivery of electrical pulses using at least one electrode of a stimulation lead;
memory configured to store program instructions; and
one or more processors that, when executing the program instructions, are configured to:
deliver the pulses to the at least one electrode, the pulses having a corresponding pulse duration and an amplitude, the amplitude varying over the pulse duration of at least one of the pulses;
monitor a signal indicative of a present level of the amplitude of the at least one pulse; and
based on the signal, declare a valid pulse based on the present level satisfying an initial criteria for a sub-duration of the pulse duration that is less than an entire pulse duration.

2. The IPG of claim 1, further comprising a current regulator circuit configured to output the signal, wherein the signal is indicative of the present level of current.

3. The IPG of claim 1, wherein the signal is indicative of the present level of voltage.

4. The IPG of claim 1, wherein the circuitry further comprises a voltage multiplier configured to generate the pulses, the one or more processors are further configured to direct the voltage multiplier to increase a voltage level utilized for generating the electrical pulses responsive to the present level not satisfying the initial criteria.

5. The IPG of claim 1, wherein the initial criteria includes at least one threshold level associated with the amplitude of the pulse, wherein the program instructions further comprise instructions for causing the one or more processors to:
responsive to the present level of the amplitude of the pulse being less than the at least one threshold level for at least a portion of the sub-duration of the pulse duration, declare the pulse invalid; and
responsive to the present level of the amplitude of the pulse being equal to or greater than the at least one threshold level for at least a portion of the sub-duration of the pulse duration, declare the pulse valid.

6. The IPG of claim 1, wherein the pulses include a first pulse that satisfies the initial criteria for the entire pulse duration, a second pulse that satisfies the initial criteria for the sub-duration but not for the entire pulse duration and a third pulse that does not satisfy the initial criteria for the sub-duration, the one or more processors configured to declare the first and second pulses to be valid and the third pulse to be invalid.

7. The IPG of claim 6, wherein the circuitry further comprises a power supply, coupled to at least one of a voltage multiplier or a control circuit, the power supply and the at least one of the voltage multiplier or control circuit configured to generate the first, second and third pulses.

8. The IPG of claim 1, wherein the instructions to monitor the signal further comprise instructions to monitor the signal for the sub-duration of the pulse duration, wherein the sub-duration is determined based on a predetermined number of clock signals or a predetermined time duration.

9. The IPG of claim 1, wherein the IPG is a neurostimulation device and the pulses represent a neurostimulation therapy.

10. The IPG of claim 1, wherein the program instructions further comprise instructions for causing the one or more processors to:
responsive to the pulse being declared valid:
do not set an error message; and
do not adjust a voltage level utilized for generating the electrical pulses.

11. The IPG of claim 1, wherein the program instructions further comprise instructions for causing the one or more processors to:
based on the signal, determine whether a voltage level utilized for generating the electrical pulses is equal to or above a voltage threshold value;
responsive to the voltage level being equal to or above the voltage threshold value, determine whether an auto-reduce feature is disabled, wherein the auto-reduce feature, when disabled, is configured to provide an indication that the voltage level is not to be reduced; and
responsive to the auto-reduce feature being disabled, do not adjust the voltage level utilized for generating the electrical pulses.

12. A computer implemented method for providing a pulsed therapy, comprising:
under control of one or more processors, in an implantable pulse generator (IPG), configured with executable instructions:
delivering electrical pulses to at least one electrode interconnected with the IPG, wherein the pulses have a corresponding pulse duration and an amplitude, the amplitude varying over the pulse duration of at least one of the pulses;
monitoring a signal indicative of a present level of the amplitude of the at least one pulse; and
based on the signal, declaring a valid pulse based on the present level satisfying an initial criteria for a sub-duration of the pulse duration that is less than an entire pulse duration.

13. The method of claim 12, wherein the signal is indicative of the present level of current output by a current regulator circuit of the IPG.

14. The method of claim 12, wherein the signal is indicative of the present level of voltage.

15. The method of claim 12, further comprising:
declaring an invalid pulse based on the present level not satisfying the initial criteria for a portion of the sub-duration of the pulse duration; and
responsive to the pulse being declared invalid, increasing a voltage level utilized by a voltage multiplier for generating the pulses.

16. The method of claim 12, further comprising:
responsive to the present level of the amplitude of the pulse being less than at least one threshold level for a portion of the sub-duration of the pulse duration, declaring the pulse invalid, the at least one threshold level being associated with the amplitude of the pulse; and
responsive to the present level of the amplitude of the pulse being equal to or greater than the at least one threshold level for the sub-duration of the pulse duration, declaring the pulse valid.

17. The method of claim 12, wherein the pulses include a first pulse that satisfies the initial criteria for the entire pulse duration, a second pulse that satisfies the initial criteria for the sub-duration but not for the entire pulse duration and a third pulse that does not satisfy the initial criteria for the sub-duration, the method further comprising declaring the first and second pulses to be valid and the third pulse to be invalid.

18. The method of claim 17, further comprising generating, utilizing a power supply coupled to at least one of a voltage multiplier or a control circuit, the first, second and third pulses.

19. The method of claim 12, further comprising:
responsive to the pulse being declared valid:
not setting an error message; and
maintaining a voltage level utilized for generating the electrical pulses.

20. The method of claim 12, further comprising:
based on the signal, determining whether a voltage level utilized for generating the electrical pulses is equal to or above a voltage threshold value;
responsive to the voltage level being equal to or above the voltage threshold value, determining whether an auto-reduce feature is disabled, wherein the auto-reduce feature, when disabled, is configured to provide an indication that the voltage level is not to be reduced; and
responsive to the auto-reduce feature being disabled, maintaining the voltage level utilized for generating the electrical pulses.
